(19) United States
(12) Patent Application Publication
(10) Pub. No.: US 2026/0257035 A1
(43) Pub. Date: Sep. 3, 2026

(54) APPARATUS FOR AUTOMATIC MULTI-SENSORY AND KINAESTHETIC STIMULATION

(71) Applicant: OREMI AI PRIVATE LIMITED, Singapore (SG)

(72) Inventors: Aik Seng YAK, Oremi (SG); Viet LE, Oremi (SG); Louis Kin Ming WONG, Oremi (SG); Rajiv ELLEPOLA, South Perth (AU)

(73) Assignee: OREMI AI PRIVATE LIMITED, Singapore (SG)

(21) Appl. No.: 18/878,576

(22) PCT Filed: Jul. 27, 2022
(86) PCT No.: PCT/SG2022/050534

§ 371 Date: Dec. 23, 2024

(51) Int. Cl. A61M 21/02; A61G 15/00; A61G 15/02; A61G 15/12; A61M 21/00

(52) U.S. Cl. CPC A61M 21/02; A61G 15/007; A61G 15/02; A61G 15/12; A61M 2021/0022; A61M 2021/0027; A61M 2021/005; A61M 2021/0066; A61M 2230/04

(57) ABSTRACT

An apparatus, means and method for supporting and providing multi-sensory and kinaesthetic stimulation for a user comprising a plurality of base joints mechanism, seat cushions, actuators, sound speakers, display lighting, sensors, Holter monitors and a computing device. The Holter monitors and sensors provide the current set of signal information of the user's bodily conditions and the environment to the apparatus' computing device. The apparatus' computing device then uses an Artificial Intelligence algorithm to interpret from the current set of the signal information together with the past sets of the information to optimize and activate the actuations of the base joints, audio speakers and lightings, and to create coordinated and rhythmic multi-sensory and kinaesthetic stimulations for the user.

### TECHNICAL FIELD

The present invention relates generally to a furniture, reclinable chair, sitting or resting apparatus. Specifically this invention relates to electronically controlled and mechanically articulable chairs or beds containing embedded sensors for providing feedback to a computer device and its methods of controlling and providing multi-sensory and kinaesthetic stimulation for the user.

### BACKGROUND ART

The need for safe and gentle methods of alleviating stress, bed sores, muscle wasting, general malaise, inactivity, boredom, bodily aches, elicits the solution in this invention of an apparatus with a method of providing passive exercise for the user by shifting of the users own weight. The apparatus in this invention further provides sitting support, resting support and a method to relax the user through multi-sensory stimulation for the aforesaid needs of the user.

Chairs or resting devices with articulation joints, actuators and automation are useful for relaxations, resting, recovery from exertions, well-being, passive exercises. Such chairs may be found in such places as homes, offices, care facilities, or gymnasiums. Generally, such devices include seat cushions with several articulation joints to recline the user. Some chairs include functions to articulate, vibrate or massage the user automatically. Specifically chairs providing passive exercises for the user are not common and are limited in features.

In a related prior embodiment, the relaxation apparatus for the user is a reclining chair. For relaxation purposes the user sits on such a stationary reclining chair, which may include a mechanical lever for control such as U.S. Pat. Nos. 9,504,327 and 9,504,328, European Patent Specification EP2995223 or automatically controlled with motors such as in patent U.S. Pat. Nos. 5,813,727, 7,063,383 and 7,832,800. However in these chairs the reclining angle selections are limited and angles are static upon selection. The reclining angles in these chairs are also limited to near flat but not beyond flat angle to curl over backward that is useful for passive exercise. These said prior art further do not have method to enable stretching of the user's muscles by design.

In a further related prior embodiment, the reclining chair induces relaxation to the user through specific passive exercises, such as U.S. Pat. No. 9,586,084 which is configured to provide specific abdominal exercise through rocking motion. The method of this prior art however is limited to abdominal exercise and does not provide entire body stretching passive exercises.

In another related prior embodiment, the reclining chair includes electric reclining control and automatic massaging functions such as U.S. Pat. Nos. 7,063,383 and 7,832,800. These prior art provide massage functions such as rubbing, squeezing or kneading, which are tactile stimulation on specific focus areas of the user's body. These prior art massage methods are different from the methods in this invention of shifting the user's own body weight to effect entire body stretching passive exercises.

The said prior art does not provide method for both forward and backward body curl passive exercises or method to alleviate bed sores or muscle wasting.

The said prior art further do not provide real time biofeedback or past data or cloud computing to compute the optimal chair articulation movement extents, speeds or time limits to avoid over-use risks such as deep vein thrombosis from extended sitting on the chair. These limitations result in sub-optimal or undesirable experiences for the user.

The said prior art further does not provide easy access to the seat of the apparatus, which is important for user with mobility limitations.

The said prior art further does not provide means of synchronization or coordination of the multi-sensory stimulation.

There exists, accordingly, a need for a solution comprising of safe and automatic methods to overcome the insufficiencies of the prior art in providing alleviation from one of aforesaid stress, bed sores, muscle wasting, general malaise, inactivity, boredom, bodily aches and combinations thereof of the user.

### SUMMARY OF INVENTION

The present invention is embodied in an apparatus in the form of an automatic chair comprising a base mechanism connected to a plurality of spine joints. The said apparatus, further contains a seat, a set of back rests and a set of leg rests connected to said plurality of spine joints. A pair of arm rests is disposed on either side of said seat. A computing device is disposed in said apparatus to control a plurality of actuators in said base mechanism and said spine joints to mechanically articulate said apparatus for supporting the user in various postures.

The said base mechanism includes said plurality of actuators to enable roll leftward, roll rightward, pitch forward and pitch backward mechanical articulations.

The said base mechanism is further attached to a set of caster wheels for apparatus mobility.

The said plurality of spine joints included said plurality of actuators connected to adjacent spines to enable a wide range of mechanical articulations of said spine joints over a wide range of angles to achieve curl upward and curl over backward articulations. The said wide range of articulations is not present in prior art.

The said seat, back rest and leg rests include a plurality of cushions. The cushions further contain at least one of selected from plurality of heating devices, sound speakers, sensors, display lighting and combinations thereof.

At least one of selected said arm rests or combinations thereof is able to slide parallel to the seat to allow for easy user accessibility to said seat.

At least one of selected said arm rests or combinations thereof further contains at least one of plurality of Holter monitors, control panel and combinations thereof.

The said computing device is connected to said control panel, said plurality of Holter monitors, said plurality of actuators and plurality of sensors, plurality of display lighting, plurality of sound speakers and plurality of heating devices.

The said computing device further is connected to the internet for data manipulation and cloud computing.

The said computing device utilizes an artificial intelligence algorithm to process inputs from the user through the control panel, signals from said Holter monitors, said sensors and said information from cloud computing to provide a means, which not present in prior art, of coordinated, rhythmic and periodic multi-sensory stimulation and kinaesthetic stimulation for a wide range of users with various levels of sensory abilities, while supporting the user on said apparatus.

The said apparatus in this invention provides a means and method to achieve at least one of curl over backward, curl upward, roll leftward, roll rightward mechanical articulations and combinations thereof. The said mechanical articulations shift the user's body weight to provide passive exercise and body stretching kinaesthetic stimulations for the user supported on said apparatus.

The said apparatus in this invention further provides a means and method to achieve at least one of coordinated, rhythmic, periodic and combinations thereof multi-sensory stimulation of at least one of audio, visual, thermal and combinations thereof and kinaesthetic stimulation, utilizing information utilizing feedback from said Holter monitors and said sensors to induce and optimize relaxation levels for the user supported on said apparatus.

Accordingly, it is the primary objective of the present invention addressing the needs of the user in alleviating stress, boredom, general malaise, and to provide relaxation, rejuvenation for the user through a method of the multi-sensory stimulation of at least one of visual, audio, thermal and combinations thereof and kinaesthetic stimulation.

It is the further objective of the present invention to provide passive exercise for alleviating from conditions such as bed sores, muscle wasting, muscle aches or muscle stiffness through a method of kinaesthetic stimulation.

It is the further objective of the present invention to provide a method of passive exercise for the user as an aid in physiotherapy or physical exercises.

It is the further objective of the present invention to provide a seat for the user as a leisure chair for sitting, resting, reading or any leisurely activities.

It is the further objective of the present invention to provide a seat for the user as a chair for desk work.

### Technical Problem

The need to support the user's body while providing automatic entire body passive exercises and kinaesthetic stimulation by the stretching the user's body in a natural way in curl upward, curl over backward, pitch forward, pitch backward, roll leftward and roll rightward body postures requires an apparatus comprising complex mechanism with computer control. Apparatus with such features is currently not present, or insufficient, in prior art.

The need to support the user's body while providing a method of effective sensory stimulation for a wide range of users with various levels of sensory abilities requires a set of multi-sensory stimulation and kinaesthetic stimulation. Such a method is currently not present, or insufficient, in prior art.

The need to support the user's body while providing a coordinated multi-sensory and kinaesthetic stimulation with bio-feedback from the user is useful and required to achieve an optimal alleviation from aforesaid stress, bed sores, muscle wasting, general malaise, inactivity, boredom, bodily aches in the user. Such a method is currently not present, or insufficient, in prior art.

### Solution to Problem

Accordingly, to solve the technical problem of a providing a means and method of automatic entire body passive exercises and kinaesthetic stimulation for the user, the apparatus in this invention utilizes a computing device, a base mechanism, a plurality of spine joints with a plurality of actuators to enable a wide angle range of automatic mechanical articulations of at least one of selected curl upward, curl over backward, pitch forward and backward, roll leftward, roll rightward and combinations thereof motions, to support the user's body and to shift the user's body weight into multiple postures.

Accordingly, to solve the technical problem of providing multi-sensory stimulation, the said apparatus in this invention provides a means to support the user's body and to provide multi-sensory stimulation through the use of a pair of audio speakers, lighting displays and heating devices. The said apparatus accordingly is capable of providing at least one of selected audio, visual, thermal and combinations thereof stimulation and kinaesthetic stimulation for the user.

Accordingly, to solve the technical problem of providing a method to coordinate the multi-sensory and kinaesthetic stimulation and to optimize alleviation from one of selected aforesaid stress, bed sores, muscle wasting, general malaise, inactivity, boredom, bodily aches and combinations thereof, the said apparatus utilizes a computing device and artificial intelligence algorithm to process inputs from the control panel, feedback signals from built-in Holter monitors and sensors, and information from cloud computing to provide an optimized and personalized set of coordinated, rhythmic and periodic multi-sensory stimulation and kinaesthetic stimulation for the user, while supporting the user in said apparatus.

### Advantageous Effects of Invention

The apparatus of the present invention provides alleviation from one of selected aforesaid stress, bed sores, muscle wasting, general malaise, inactivity, boredom, bodily aches and combinations thereof in a safe and gentle method through shifting the users own body weight. The advantage of shifting the user's body weight is that it provides entire body passive exercise through natural stretching, which is less of an intervention on the body as compared to compression or depression on focused areas on the body as commonly deployed in massage chairs in prior art.

The apparatus of the present invention, further provides multi-sensory stimulation and kinaesthetic stimulation to enable the use by a wide range of users with various levels of sensory abilities. The multi-sensory stimulation and kinaesthetic stimulation serves to guide the user into relaxation, rejuvenation, and enjoyment modes, which are useful for alleviating the user from boredom and stress, and to provide quality of life for the user.

The apparatus of the present invention, further utilizes bio-feedback from Holter monitors and sensors and artificial intelligence algorithms to provide multi-sensory stimulation and kinaesthetic stimulation in coordinated, rhythmic and periodic patterns. The advantage of such a user bio-feedback based and artificial intelligence based method is it achieves relaxation levels in a personal, unique and optimal method for the user.

The apparatus of the present invention, further provide the user a means of bodily support, rest or entertainment in unique and personalized postures.

The apparatus of the present invention, further provide the user with said visual display lighting feedback of the user's Holter monitor status to guide the user in a unique and personalized way into deeper relaxation levels for sense of enjoyment or personal achievement.

### BRIEF DESCRIPTION OF DRAWINGS

The following description of the accompanying figures are exemplifications of a preferred embodiment of the present invention, and it does not limit the present invention, with the understanding that the preferred embodiment in the present disclosure is an exemplification of the present invention.

FIG. 1 Illustrates the architecture of the apparatus in the present invention in accordance with a preferred embodiment from a side view including some of its main components in the view.

FIG. 2 Illustrates the architecture of the apparatus in the present invention in accordance with a preferred embodiment from a front isometric view including some of its main components in the view.

FIG. 3 Illustrates the architecture of the apparatus in the present invention in accordance with a preferred embodiment in a back isometric view including some of its main components in the view.

FIG. 4 Illustrates the architecture of the apparatus in the present invention in accordance with a preferred embodiment from a top view including some of its main components in the view.

FIG. 5 Illustrates a use case exemplification, in a front isometric view, of a user utilizing the apparatus in the present invention in accordance with a preferred embodiment, in an exemplification of a supported body posture brought about by a mechanical articulation configuration of said apparatus.

FIG. 6 Illustrates a curl over backward mechanical articulation configuration exemplification of the apparatus in the present invention in accordance with a preferred embodiment.

FIG. 7 Illustrates a combination of curl upward and pitch backward mechanical articulation configuration exemplification of the apparatus in the present invention in accordance with a preferred embodiment.

FIG. 8 Illustrates the sliding arm rest configuration of the apparatus in the present invention in accordance with a preferred embodiment.

FIG. 9 Illustrates a user case exemplification of a user utilizing the apparatus in the present invention in accordance with a preferred embodiment, in a curl over backward supported body posture brought about by a curl over backward mechanical articulation configuration of said apparatus.

FIG. 10 Illustrates a user case exemplification of a user utilizing the apparatus in the present invention in accordance with a preferred embodiment, in a combination of curl upward and pitch backward body posture brought about by a combination of curl upward and pitch backward mechanical articulation configuration in said apparatus.

FIG. 11 Illustrates a combination of curl upward, pitch backward and roll leftward mechanical articulation configuration exemplification of the apparatus in the present invention in accordance with a preferred embodiment.

FIG. 12 Illustrates the schematic of the preferred method in the present invention.

### DESCRIPTION OF EMBODIMENTS

The following detailed description of the accompanying figures are exemplifications for the preferred embodiments of the apparatus in the present invention, and it is not intended to not limit the present invention, with the understanding that the preferred embodiment in the present disclosure is an exemplification of the present invention.

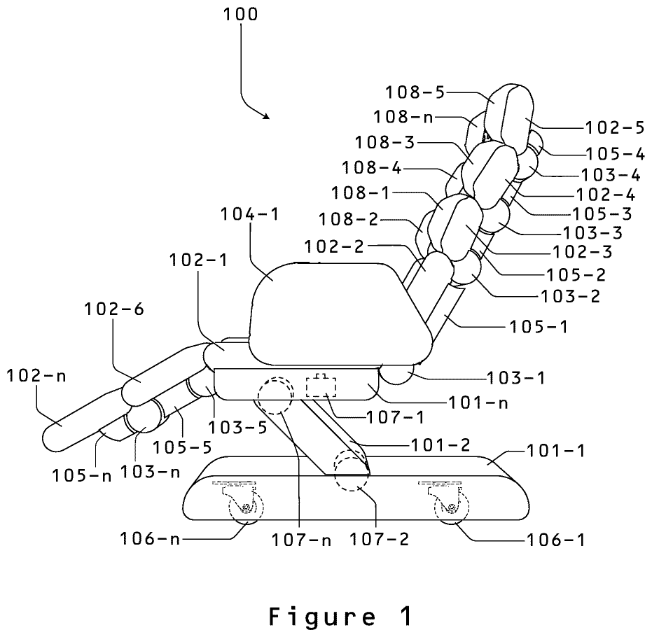

The preferred embodiment of the apparatus in the present invention is represented in FIG. 1-FIG. 12. The preferred embodiment of the apparatus in the present invention will hereinafter be referred to as the apparatus 100.

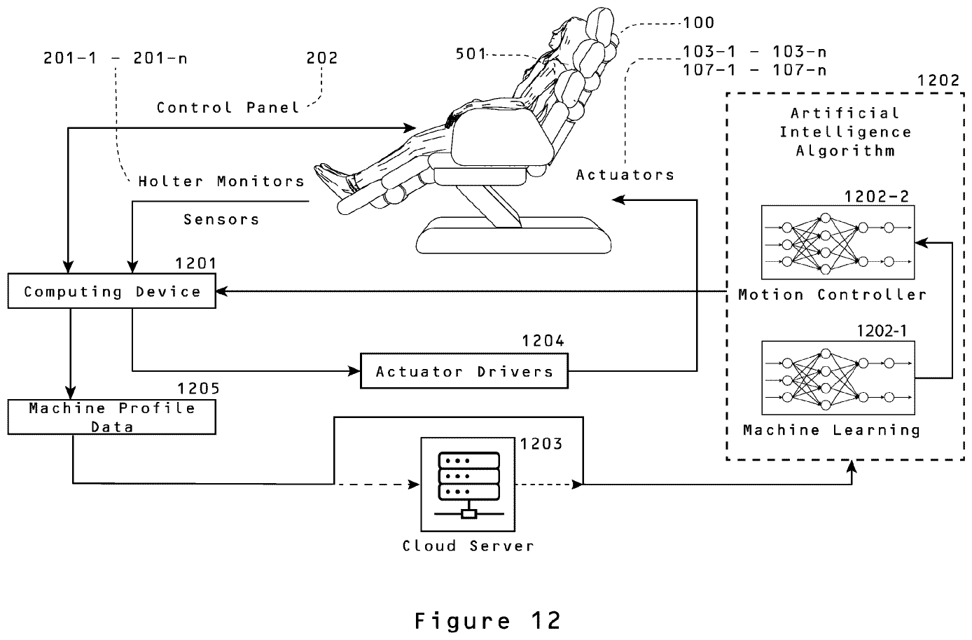

The preferred embodiment of the method in said apparatus 100 of the present invention is represented in FIG. 12.

The said apparatus 100 in the form of an automatic chair comprising a base mechanism 101-1-101-n connected to a plurality of spine joints 105-1-105-n. The said apparatus 100, further contains a plurality of seat, back rests and leg rests cushions 102-1-102-n connected to said plurality of spine joints. A pair of arm rests 104-1-104-2 is disposed on either side of said seat. A computing device 1201 is disposed in said apparatus 100 to control a plurality of actuators in said base mechanism 101-1-101-n and said spine joints 105-1-105-n to mechanically articulate said apparatus 100 for supporting the user in various postures.

The said base mechanism 101-1-101-n includes base 101-1, base link 101-2 and base seat 101-n connected to each other as exemplified in FIG. 1.

The said base 101-1, further is designed to sit on a ground surface.

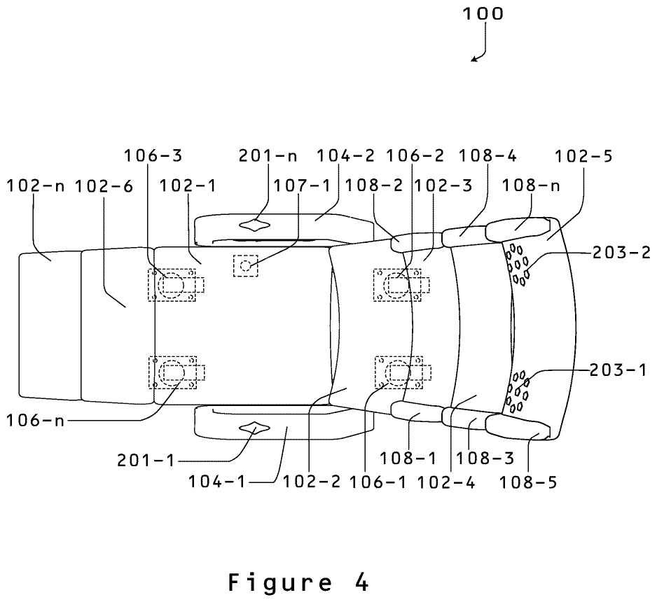

The said base 101-1, further is attached to a plurality of caster wheels 106-1-106-n to enable mobility of said apparatus, as exemplified in FIG. 1 and FIG. 4.

The said plurality of caster wheels 106-1-106-n, further includes lockable feature to keep the wheels from rotating and to keep the apparatus in place during operation.

The said base mechanism 101-1-101-n, further comprised a plurality of base actuators 107-1-107-n to enable mechanical articulations of roll leftward, roll rightward, pitch forward and pitch backward mechanical articulations of said apparatus 100. Said base actuator 107-1, as exemplified in FIG. 1 and FIG. 4, is utilized for said roll leftward and roll rightward mechanical articulations of said apparatus 100. At least one of said base actuators 107-2, 107-n and combinations thereof, as exemplified in FIG. 1, is utilized to enable mechanical articulation of said pitch forward and pitch backward of said apparatus 100.

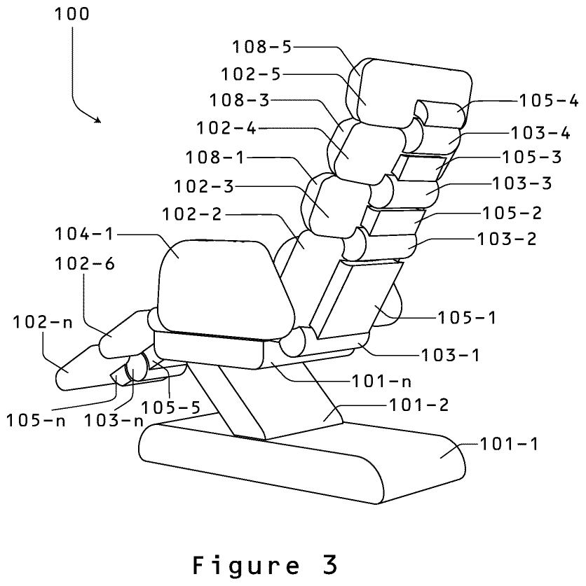

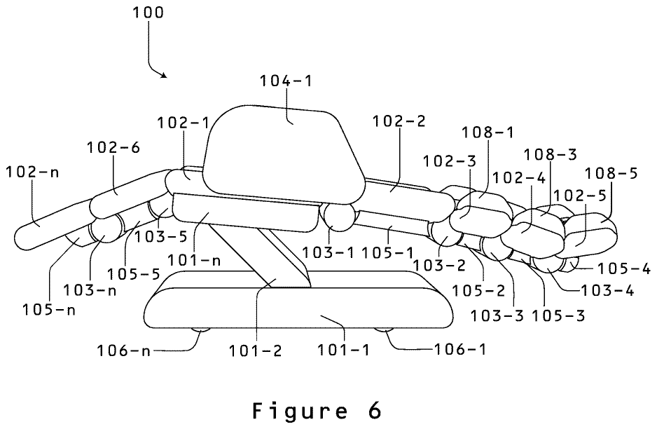

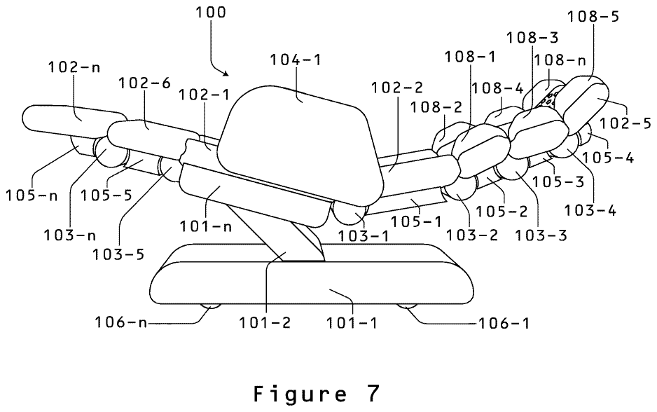

The said plurality of spine joints 105-1-105-n included said plurality of spine actuators 103-1-103-n, connected to each adjacent spine joints 105-1, 105-2, 105-3, 105-4, 105-5 and 105-n, as exemplified in FIG. 1 and FIG. 3, to enable a wide range of mechanical articulations of said spine joints over a wide range of angles to achieve curl upward and curl over backward articulations, as exemplified in FIG. 6 and FIG. 7.

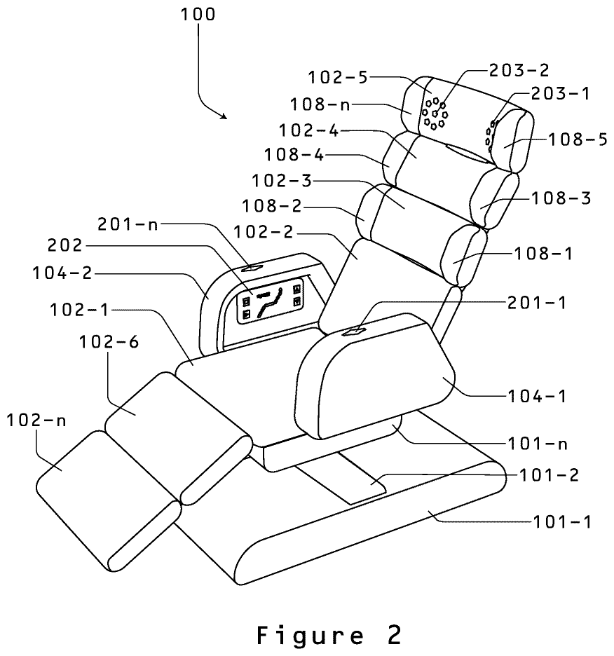

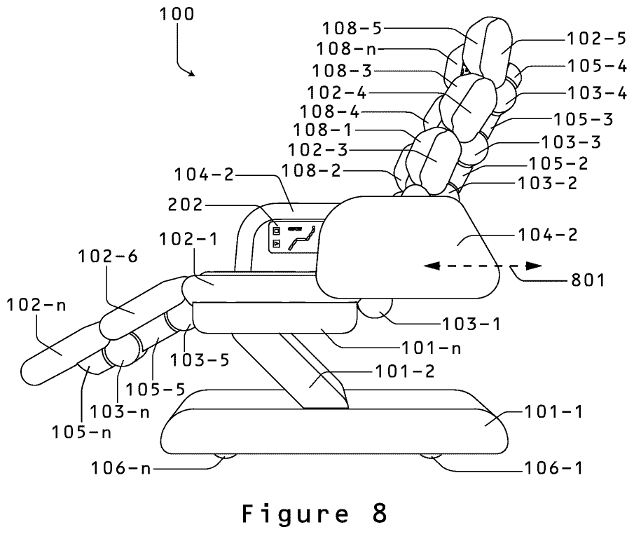

The said arm rests 104-1-104-2, referring to FIG. 2, contains at least one of selected the plurality of Holter monitors 201-1-201-n, a plurality sensors, and combinations thereof, and a control panel 202. The said arm rests 104-1-104-2 has the ability to slide along sideways 801 along the seat cushion 102-1, as exemplified in FIG. 8, to allow for easy accessibility for the user 501.

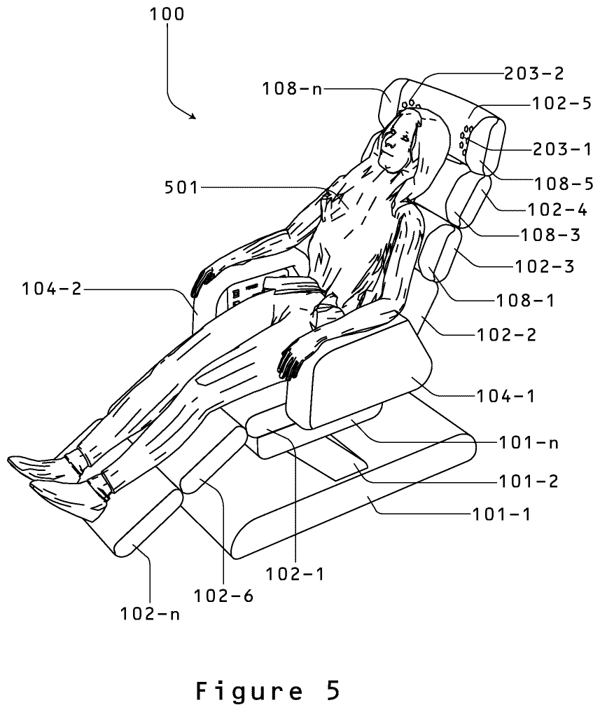

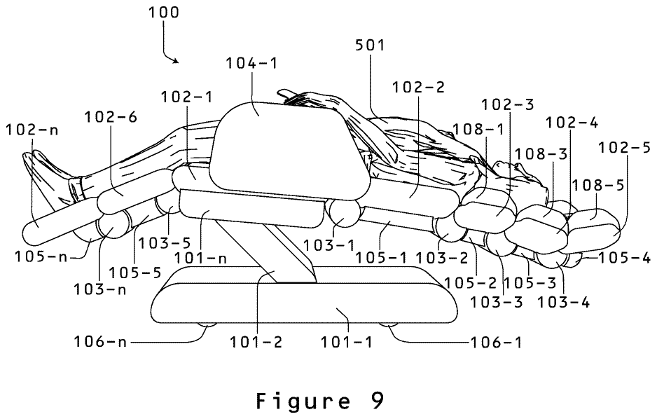

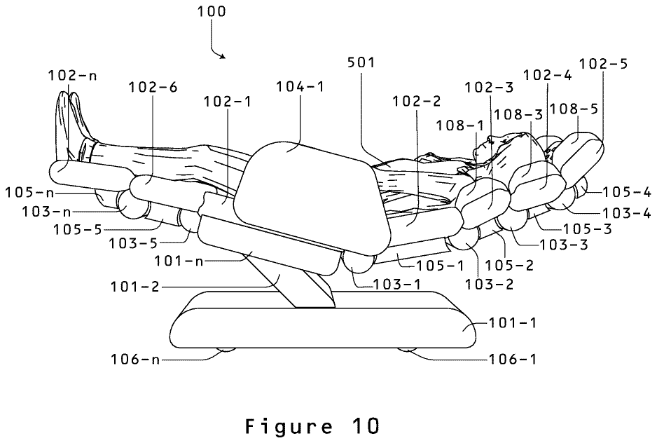

The said plurality of cushions 102-1-102-n, referring to FIG. 2 and FIG. 4, contain cushion shape with slight curve profiles to provide comfort to the user and to support the user 501 body securely during various said mechanical articulation of said apparatus 100. FIG. 5, FIG. 9 and FIG. 10 show exemplifications of how a user 501 is supported in said apparatus 100.

The said cushions 102-3-102-5 for supporting the upper body of the user 501, further comprising high profile cushion as a privacy feature and to secure the body better during said mechanical articulation of said apparatus 100.

The said cushions 102-3-102-5 for supporting the upper body of the user 501, further contains at least one of selected plurality of lighting display 108-1-108-n each comprising multiple LED colors and combinations thereof, as exemplified in FIG. 1-FIG. 11. The said plurality of lighting 108-1-108-n display show the interpreted information from the plurality of Holter monitors 201-1-201-n including at least one of the user's 501 heart rate, heart rate variability and combinations thereof as a form of biofeedback to the user 501. The said plurality of lighting display 108-1-108-n, further provides a means to indicate an urgent need for attention to an external party.

The said plurality of light display 108-1-108-n, further each contains at least one of Red, Green, Amber, White color LEDs and combinations thereof and enables the user to personalize the colors and combinations of colors.

The said cushion 102-5 for supporting the top upper body of the user 501, further contains a pair of audio speakers 203-1-203-2 to enable sound projection to the user 501 in coordination with the kinaesthetic stimulation afforded by said mechanical articulations of said apparatus 100.

At least one of selected of said plurality of cushions 102-1-102-n and combinations thereof, further contain heating device to provide a means of thermal sensory stimulation for the user 501. Such said thermal stimulation is useful to assist and alleviate the user during body recovery, relaxation and rejuvenation.

The said plurality of cushions 102-1-102-n, further are connected to the plurality of spine joints 105-1-105-n and are each articulable by at least one of selected plurality of spine actuators 103-1-103-n, plurality of base actuators 107-1-107-n and combinations thereof.

The said plurality of spine actuators 103-1-103-n and plurality of base actuators 107-1-107-n, further are powered by at least one of selected electric, pneumatic, hydraulic and combinations thereof.

The said plurality of spine actuators 103-1-103-n and plurality of base actuators 107-1-107-n, further may be individually powered or powered as part of a linkage.

The control panel 202 includes a set of tactile buttons connected to said computer device 1201 and provides the means for the user 501 to set the static and dynamic configurations and motions of said mechanical articulations of the apparatus.

The control panel 202, further provides the user 501 a means to Start, Pause and Stop the operation of said apparatus 100.

The mechanical articulation of said apparatus 100 is able to curl over backward, as exemplified in FIG. 6, by utilizing said plurality of spine actuators 103-1-103-n to articulate the said plurality of cushions 102-1-102-n.

The mechanical articulation of said apparatus 100, further is able to curl upward, as exemplified in FIG. 7, by utilizing said plurality of spine actuators 103-1-103-n to articulate the said plurality of cushions 102-1-102-n.

The mechanical articulation of said apparatus 100, further is able to pitch forward and backward by utilizing said plurality of base actuators 107-1-107-n to articulate said plurality of spine joints 105-1-105-n. FIG. 7 is an exemplification of a combination of pitch backward and curl upward mechanical articulation of said apparatus 100.

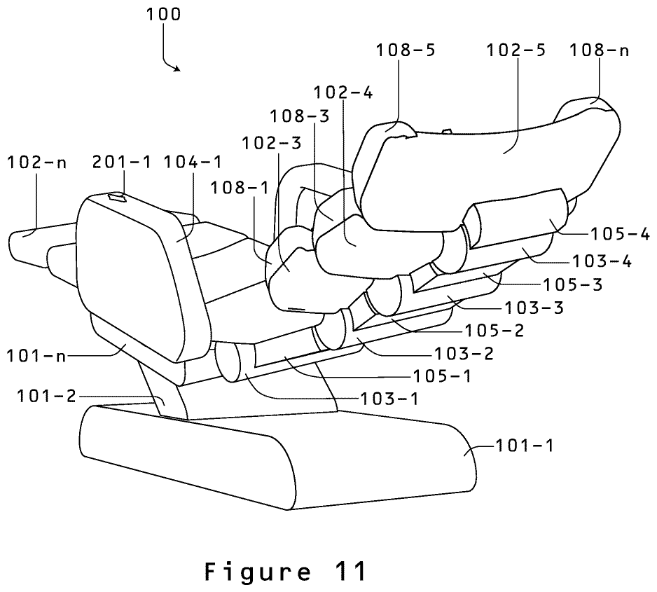

The mechanical articulation of said apparatus 100, further is able to roll leftward and roll rightward by utilizing said plurality of base actuators 107-1-107-n to articulate said plurality of spine joints 105-1-105-n. FIG. 11 is an exemplification of a combination of pitch backward, curl upward and roll leftward mechanical articulation of said apparatus 100.

Accordingly, with the capabilities of aforesaid mechanical articulations, the said apparatus 100 is able to provide varying degrees of mechanical articulations of said apparatus 100 to bring about corresponding posture change in the user 501 while supporting said user 501 in the apparatus. FIG. 9 and FIG. 10 are exemplifications of two of the multiple variations in possible articulation and posture.

Referring to the schematic in FIG. 12 of the preferred method, the said apparatus utilizes an onboard said computing device 1201 as a central part of the method to compute and control the operation of said apparatus to provide coordinated said multi-sensory stimulation and kinaesthetic stimulation for the user 501.

The said computing device 1201 is connected to the control panel 202 to receive direct commands and information from the user 501.

The said computing device 1201, is further connected to the plurality Holter monitors 201-1-201-n and sensors, to receive biofeedback signals from the said Holter monitors 201-1-201-n and sensors, for interpretation of the status of the user 501 utilizing the apparatus. The said the status of the user 501 from the said Holter monitors 201-1-202-n and sensors includes at least one of selected heart rate, heat rate variability, body temperature, body weight and combinations thereof. The said interpretation of the status of the user 501 from the biofeedback signals include relaxation levels, cardiovascular reactions and well-being of the user 501.

The said computing device 1201, is further connected to the actuator drivers 1204 of the said plurality of base actuators 107-1-107-n and said plurality of spine actuators 103-1-103-n, to control the motions of the actuators for enabling multiple rhythmic and periodic motion configurations of said mechanical articulations of said apparatus 100.

The said computing device 1201 utilizes a method of artificial intelligence algorithm 1202 to interpret the status of the user 501 from biofeedback signals to determine the desired configurations of the rhythmic and periodic motion configurations of said mechanical articulations, to bring about passive exercises and relaxation of the user 501 while supported on said apparatus 100.

The said computing device 1201, is further connected to the pair of audio speakers 203-1-203-2 to provide audio projection in coordination with the desired rhythmic and periodic said mechanical articulation configurations, determined by the artificial intelligence algorithm 1202, to guide the user 501 for a regulated breathing, body motion and desired relaxation status.

The said computing device 1201, is further connected to a cloud server 1203, either on the internet or in a separate network, to enable uploading and downloading of historical data, present data and pooled data, and to enable artificial intelligence based data mining and cloud computing. The information and interpretation from the cloud server 1203 is utilized in said computing device to aid in determining the type, limits and configuration of multi-sensory stimulation and kinaesthetic stimulation to provide to the user 501 in said apparatus during operation.

The said computing device 1201, further provides a means to limit the duration of usage by the user 501 in said apparatus 100 to within a safe time period between each usage. This said safe time period is personalized and determined by the user 501 to limit said multi-sensory stimulation and said kinaesthetic stimulation, and is guided by the user's age group and health conditions.

The said computing device 1201, further provides a means to limit the physical range of said mechanical articulations by the user 501 in said apparatus 100 to within a safe range of articulation angles and configurations. This said safe range of articulation angles and configurations is personalized and determined by the user 501, and is guided by the user's age group and health conditions.

The said safe time period, said safe range of articulation angles and configurations, age and usage related information from the user are stored locally and in the cloud server 1203 as machine profile data 1205, to enable artificial intelligence algorithm 1202 based machine learning 1202-1 and motion control 1202-2 of said apparatus 100.

In another preferred embodiment, the computing device 1201, is incorporated with computer generated voice and connected to microphone and infrared sensors. The computing device 1201 is able to detect users around the chair and to interpret voice command from the user 501. An exemplification a reminder from said apparatus to the user 501 to experience the relaxation session at specific time of the day, “Good morning Julia, would you like to have relaxation session now?”. Another exemplification is a reminder message sent via internet, “It is now approaching 7 pm, don't forget to call Janet” or “You have an appointment with Dr. James at 8 am tomorrow”.

Accordingly, the said apparatus 100 of the present invention, has the capability and features to provide user customizable preferred at least one of static posture, periodic motions and combinations thereof for various needs or intent. An exemplification of a preferred periodic motion for the user 501 is of gentle rocking with at least one of pitching forward and backward, rolling left and right, or combinations thereof for the intention of inducing sleep or pleasure. An exemplification of a preferred static posture for the user 501 is of a combination of curl upward and pitch backward posture for the intention of reading or watching television. A further exemplification is of a preferred combination of static posture and periodic motion for the user 501, which enables a predominantly static posture for the user 501 but with occasional automatic mechanical articulation to shift the posture of the user 501, to encourage occasional body movements of the user 501 supported on said apparatus. The control panel allows the user 501 to set the motion or posture configuration in these exemplifications.

Accordingly, the said apparatus 100 of the present invention is a unique form of apparatus with the ability to provide a means and method of automatic entire body passive exercises and kinaesthetic stimulation in a wide range of supported body posture, in a safe and natural way for the user 501, to alleviate the user 501 from stress, bed sores, muscle wasting, general malaise, inactivity, boredom, bodily aches.

Accordingly, the said apparatus 100 of the present invention, further is a unique form of apparatus with the ability to provide a method of coordinated multi-sensory stimulation and kinaesthetic stimulation, utilizing biofeedback, artificial intelligence algorithm 1202 and cloud computing, to enable the use by a wide range of users with various levels of sensory abilities, to guide the user into relaxation, rejuvenation, and enjoyment modes, which are useful for alleviating the user from boredom and stress, and to provide quality of life for the user.

### CITATION LIST

PTL1: U.S. Pat. No. 9,504,327; Ledat

PTL2: U.S. Pat. No. 9,504,328; Garland

PTL3: U.S. Pat. No. 5,813,727; Sugawa et al.

PTL4: U.S. Pat. No. 7,063,383; Wu

PTL5: U.S. Pat. No. 7,832,800; Lo

PTL6: U.S. Pat. No. 9,586,084; Duke

PTL7: US Patent Literature

PTL8: EP2995223 Bl; Lin, Chang-Chen

### Non Patent Literature

NPL1: Mutsuhiro Nakao, “Heart Rate Variability and Perceived Stress as Measurements of Relaxation Response”, J Clin Med. 2019 October; 8(10):1704.

NPL2: Willeke Van Dijk, Anja C. Huizink, Jasmin Muller, Kerstin Uvnas-Moberg, Anette Ekstrom-Bergstrom and Linda Handlin, “The Effect of Mechanical Massage and Mental Training on Heart Rate Variability and Cortisol in Swedish Employees—A Randomized Explorative Pilot Study”, Front. Public Health, Mar. 19, 2020.

I claim

1. An apparatus for supporting and stimulating a user understanding:
a base mechanism comprising a plurality of base joints to support said apparatus and to enable mechanical articulation of said apparatus;
a plurality of spine joints attached above the said base mechanism to enable mechanical articulation of said apparatus;
a plurality of cushions which are arranged in said plurality of spine joints mechanism;
a pair of arm rests disposed on both sides of said apparatus;
a plurality of actuators to enable powered or automatic movements of said base mechanism and said plurality of spine joints;
a plurality of sound speakers disposed in at least one selected from said plurality of cushions and combinations thereof;
a plurality of display lighting disposed in at least one selected from said plurality of cushions and combinations thereof;
a plurality of sensors, including at least one of microphone, infrared detector, weight sensor and combinations thereof, disposed in at least one selected from said plurality of cushions and combinations thereof;
a plurality of Holter monitors disposed in at least one selected from said pair of arm rests and combinations thereof;
a plurality of heating devices disposed in at least one of selected from said plurality of cushions and combinations thereof;
a control panel for control of the apparatus disposed on at least one of selected pair of arm rests and combinations thereof;
a computing device for reading and computing information from at least one of said plurality of sensors and plurality of Holter monitors and combinations thereof and controlling said plurality of actuators.

2. A method wherein said apparatus as set forth in claim 1, utilizes said computing device and at least one of selected from said plurality of actuators and said plurality of spine joints and combinations thereof to coordinate the motions of said plurality of cushions to enable combinations of articulations of the user's body posture while supported in said apparatus.

3. The method as claimed in claim 2 wherein said user's body posture is curled over backward while supported on said apparatus.

4. The method as claimed in claim 3 wherein said combinations of articulations prevent the user from sliding backward during said curled over backward posture while supported on said apparatus.

5. The method as claimed in claim 2 wherein said user's body posture is curled upward so that both the user's torso and legs tilt upward while supported on said apparatus.

6. A method wherein the apparatus as set forth in claim 1 utilizes said computing device and at least one of selected from said plurality of actuators and said base mechanism and said plurality of cushions and combinations thereof to roll leftward and roll rightward the user while supported on said apparatus.

7. The method as claimed in claim 6 wherein the user's body is tilted in at least one of selected backward pitch, forward pitch and combinations thereof while supported on said apparatus.

8. A method wherein the apparatus as set forth in claim 1 utilizes at least one of said plurality of Holter monitors and said plurality of sensors and combinations thereof to provide at least one of selected current set of the user's bodily conditions information and the environment condition information and combinations thereof to said computing device.

9. The method as claimed in claim 8 wherein said computing device utilizes at least one of said bodily conditions information and said environmental condition information and combinations thereof to compute and coordinate the control of said plurality of actuators to articulate the user's posture in combinations of periodic motions while supported on said apparatus.

10. The apparatus as set forth in claim 1, wherein at least one of said plurality of display lighting or combinations thereof provide a means of visual display of user's Holter monitor information for visual feedback to the user while supported on said apparatus.

11. The method as claimed in claim 9 wherein at least one of said plurality of sound speakers and combinations thereof provide a means of sound projection in coordination with said combinations of periodic motions for the user while supported on said apparatus.

12. The method as claimed in claim 9, wherein at least one of said plurality of heated devices or combinations thereof provide a means of thermal stimulation during said combinations of periodic motions for the user while supported on the apparatus.

13. The apparatus as set forth in claim 1, wherein at least one of said pair of arm rests and combinations thereby enables a method of accessibility to the seat of said apparatus for the user.

14. A method wherein said apparatus as set forth in claim 1 utilizes said computing device to provide computer generated voice through said plurality of sound speakers, and together with at least one of selected said plurality of sensors or combinations thereof on said apparatus to detect and to communicate with the user.
